(19) United States
(12) Patent Application Publication
(10) Pub. No.: US 2026/0224188 A1
(43) Pub. Date: Aug. 6, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Tsuyoshi MATSUMOTO, Kanagawa (JP)

(73) Assignee: FUJIFILM Corporation, Tokyo (JP)

(21) Appl. No.: 19/437,354

(22) Filed: Dec. 31, 2025

(30) Foreign Application Priority Data
Feb. 6, 2025 (JP) 2025-018227

(51) Int. Cl. A61B 8/08; A61B 8/00; G06T 7/62

(52) U.S. Cl. CPC A61B 8/085; A61B 8/0883; A61B 8/461; A61B 8/469; A61B 8/5223; A61B 8/54; G06T 7/62; G06T 2207/10132; G06T 2207/30048

(57) ABSTRACT

Provided are an ultrasound diagnostic apparatus and a control method of an ultrasound diagnostic apparatus that enable a user to accurately and easily dispose a measurement line at an appropriate position.

An ultrasound diagnostic apparatus includes an image recognition unit that performs image recognition of a local anatomical structure of a heart from an ultrasound image, a measurement line generation unit that generates a measurement line, a measurement reference point setting unit that sets a measurement reference point within a predetermined appropriate range of the measurement line in the ultrasound image based on the appropriate range and the local anatomical structure subjected to the image recognition, a display form setting unit that sets a display form of the measurement line based on a positional relationship with respect to the measurement reference point, a monitor, and a display controller that displays the measurement line on the monitor in accordance with the set display form.

### CROSS-REFERENCE TO RELATED APPLICATIONS

The present application claims priority under 35 U.S.C. § 119 to Japanese Patent Application No. 2025-018227, filed on Feb. 6, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a control method for the ultrasound diagnostic apparatus.

### 2. Description of the Related Art

In the related art, a so-called cardiac output is calculated by capturing an ultrasound image representing a tomographic plane of a heart of a subject using a so-called ultrasound diagnostic apparatus and analyzing the captured ultrasound image. The cardiac output is usually calculated by a calculation step of (1) measuring a diameter of a left ventricular outflow tract in an ultrasound image of a frame representing a so-called parasternal left ventricular long-axis cross section at a mid-systolic phase of the heart to calculate a cross-sectional area of the left ventricular outflow tract, (2) calculating a velocity time integral value of blood flow in the left ventricular outflow tract with respect to a so-called apical five-chamber cross section or a so-called apical three-chamber cross section by a so-called pulse Doppler method, (3) calculating a so-called stroke volume by multiplying the cross-sectional area of the left ventricular outflow tract by the velocity time integral value of the blood flow in the left ventricular outflow tract, and (4) calculating the cardiac output by multiplying the stroke volume by a heart rate.

In a case in which the diameter of the left ventricular outflow tract is measured in the calculation of the cardiac output, a measurement line may be disposed on the left ventricular outflow tract in the ultrasound image, and a length of the measurement line may be measured as the diameter of the left ventricular outflow tract. The appropriate range for a disposition position of the measurement line is often determined by a guideline of an examination established by a public interest organization such as an academic society or a guideline predetermined by institutional guidelines of a facility such as a hospital in which the examination is performed. However, in particular, a user who is not skilled in the examination may have difficulty in determining whether or not a position of the measurement line disposed by the user is within the appropriate range. Therefore, in order for the user to easily understand whether or not the position of the measurement line is within the appropriate range, for example, a technique disclosed in JP2015-156960A is considered. JP2015-156960A discloses specifying a position of an aortic valve annulus and highlighting the position based on a brightness of an ultrasound image. The appropriate range for the position of the measurement line is often set based on the aortic valve annulus. Therefore, the user can dispose the measurement line while checking the highlighted position of the aortic valve annulus.

### SUMMARY OF THE INVENTION

Incidentally, according to the established guidelines, for example, a specific position within an appropriate range may be determined as the optimum position of the measurement line. Even in a case in which the user can check the position of the aortic valve annulus by the technique of JP2015-156960A, the user cannot accurately understand a positional relationship between a set position of the measurement line and an appropriate position, and cannot dispose the measurement line at the appropriate position.

The present invention has been made to solve such a problem in the related art, and an object of the present invention is to provide an ultrasound diagnostic apparatus and a control method of an ultrasound diagnostic apparatus that enable a user to accurately and easily dispose a measurement line at an appropriate position.

The above object can be achieved with the following configurations.
[1] An ultrasound diagnostic apparatus that displays a measurement line of a left ventricular outflow tract diameter in an ultrasound image in which a heart of a subject is imaged, the ultrasound diagnostic apparatus comprising:
   an image recognition unit that performs image recognition of a local anatomical structure of the heart from the ultrasound image;
   a measurement line generation unit that generates the measurement line;
   a measurement reference point setting unit that sets a measurement reference point within a predetermined appropriate range of the measurement line in the ultrasound image based on the appropriate range and the local anatomical structure subjected to the image recognition by the image recognition unit;
   a display form setting unit that sets a display form of the measurement line generated by the measurement line generation unit based on a positional relationship with respect to the measurement reference point set by the measurement reference point setting unit;
   a monitor; and
   a display controller that displays the measurement line on the monitor in accordance with the display form set by the display form setting unit.
[2] The ultrasound diagnostic apparatus according to [1], further comprising:
   a memory that stores the appropriate range.
[3] The ultrasound diagnostic apparatus according to [1] or [2],
   in which the appropriate range is a range reflecting a preference of a user or a range defined by an established guideline.
[4] The ultrasound diagnostic apparatus according to any one of [1] to [3],
   in which the image recognition unit performs image recognition of any of an aortic valve, a mitral valve, and a left ventricle as the local anatomical structure.
[5] The ultrasound diagnostic apparatus according to any one of [1] to [4],
   in which the measurement line generation unit generates the measurement line based on the local anatomical structure subjected to the image recognition by the image recognition unit.
[6] The ultrasound diagnostic apparatus according to any one of [1] to [5],
   in which the measurement reference point setting unit sets a midpoint of the appropriate range as the measurement reference point.
[7] The ultrasound diagnostic apparatus according to any one of [1] to [5],
   in which the measurement reference point setting unit sets the measurement reference point in accordance with a preference of a user based on a past measurement result.
[8] The ultrasound diagnostic apparatus according to any one of [1] to [7],
   in which the display form setting unit sets a display form in which at least one of a color, a shape, or a type of the measurement line is changed depending on the positional relationship of the measurement line with respect to the measurement reference point.
[9] The ultrasound diagnostic apparatus according to any one of [1] to [6], further comprising:
   an input device that receives an input operation by a user,
   in which the measurement reference point setting unit sets the measurement reference point based on the appropriate range input via the input device and the local anatomical structure subjected to the image recognition by the image recognition unit.
[10] The ultrasound diagnostic apparatus according to [2], further comprising:
   a user recognition unit that recognizes a user,
   in which the memory stores a plurality of appropriate ranges corresponding to a plurality of users, and
   the measurement reference point setting unit reads out the appropriate range corresponding to the user recognized by the user recognition unit from the memory, and sets the measurement reference point based on the read appropriate range.
[11] The ultrasound diagnostic apparatus according to [10], further comprising:
   an input device that receives an input operation by the user,
   in which the user recognition unit recognizes the user based on user identification information input via the input device.
[12] The ultrasound diagnostic apparatus according to [10],
   in which the user recognition unit recognizes the user by using biometric authentication.
[13] A control method of an ultrasound diagnostic apparatus that displays a measurement line of a left ventricular outflow tract diameter in an ultrasound image in which a heart of a subject is imaged, the control method comprising:
   performing image recognition of a local anatomical structure of the heart from the ultrasound image;
   generating the measurement line;
   setting a measurement reference point within a predetermined appropriate range of the measurement line in the ultrasound image based on the appropriate range and the local anatomical structure subjected to the image recognition;
   setting a display form of the measurement line based on a positional relationship with respect to the measurement reference point; and
   displaying the measurement line on a monitor in accordance with the set display form.

In the ultrasound diagnostic apparatus according to the present invention, the ultrasound diagnostic apparatus comprises an image recognition unit that performs image recognition of a local anatomical structure of the heart from the ultrasound image, a measurement line generation unit that generates the measurement line, a measurement reference point setting unit that sets a measurement reference point within a predetermined appropriate range of the measurement line in the ultrasound image based on the appropriate range and the local anatomical structure subjected to the image recognition by the image recognition unit, a display form setting unit that sets a display form of the measurement line generated by the measurement line generation unit based on a positional relationship with respect to the measurement reference point set by the measurement reference point setting unit, a monitor, and a display controller that displays the measurement line on the monitor in accordance with the display form set by the display form setting unit. Thereby, the user can accurately and easily dispose the measurement line at the appropriate position.

### BRIEF DESCRIPTION OF THE DRAWINGS

FIG. 1 is a block diagram showing a configuration of an ultrasound diagnostic apparatus according to First Embodiment of the present invention.

FIG. 2 is a block diagram showing an internal configuration of a transmission/reception circuit in First Embodiment of the present invention.

FIG. 3 is a block diagram showing an internal configuration of an image generation unit in First Embodiment of the present invention.

FIG. 4 is a diagram showing an example of an ultrasound image representing a parasternal left ventricular long-axis cross section.

FIG. 5 is a diagram schematically showing an example of a measurement line disposed on a left ventricular outflow tract of a heart in the ultrasound image.

FIG. 6 is a diagram schematically showing an example of a reference point set on the left ventricular outflow tract of the heart and an appropriate range for a disposition position of the measurement line in the ultrasound image.

FIG. 7 is a diagram schematically showing an example of display forms of the measurement line that are different according to a positional relationship with respect to the reference point.

FIG. 8 is a flowchart showing an operation of the ultrasound diagnostic apparatus according to First Embodiment of the present invention.

FIG. 9 is a block diagram showing a configuration of an ultrasound diagnostic apparatus according to Second Embodiment of the present invention.

### DESCRIPTION OF THE PREFERRED EMBODIMENTS

Hereinafter, embodiments of the present invention will be described based on the accompanying drawings.

The following configuration requirements are described based on a representative embodiment of the present invention, but the present invention is not limited to the embodiment.

In the present specification, a numerical range represented by “to” means a range including numerical values described before and after “to”, both ends inclusive, as a lower limit value and an upper limit value.

In the present specification, “the same” includes an error range generally allowed in the technical field.

### FIRST EMBODIMENT

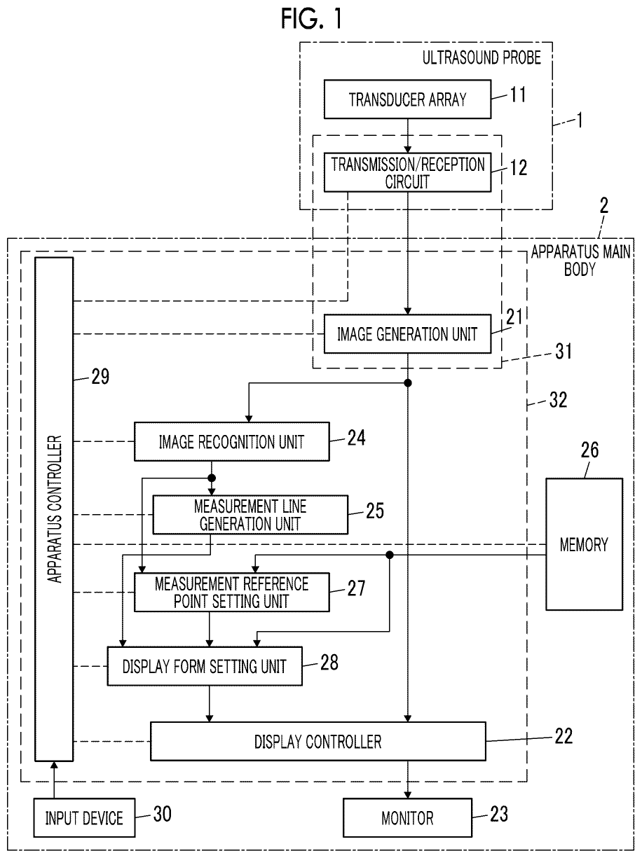

FIG. 1 shows a configuration of an ultrasound diagnostic apparatus according to First Embodiment of the present invention. The ultrasound diagnostic apparatus comprises an ultrasound probe 1 and an apparatus main body 2 that are connected to each other by so-called wired communication or so-called wireless communication.

The ultrasound probe 1 comprises a transducer array 11 and a transmission/reception circuit 12 connected to the transducer array 11.

The apparatus main body 2 comprises an image generation unit 21 connected to the transmission/reception circuit 12. In the apparatus main body 2, a display controller 22 and a monitor 23 are sequentially connected to the image generation unit 21. In addition, an image recognition unit 24 is connected to the image generation unit 21. A measurement line generation unit 25 is connected to the image recognition unit 24. In addition, the apparatus main body 2 comprises a memory 26. A measurement reference point setting unit 27 is connected to the image recognition unit 24 and the memory 26. A display form setting unit 28 is connected to the measurement line generation unit 25, the memory 26, and the measurement reference point setting unit 27. The display form setting unit 28 is connected to the display controller 22. In addition, an apparatus controller 29 is connected to the transmission/reception circuit 12, the image generation unit 21, the display controller 22, the image recognition unit 24, the measurement line generation unit 25, the memory 26, the measurement reference point setting unit 27, and the display form setting unit 28. An apparatus controller 29 is connected to an input device 30.

The transmission/reception circuit 12 and the image generation unit 21 constitute an image acquisition unit 31. In addition, a processor 32 for the apparatus main body 2 is configured by the image generation unit 21, the display controller 22, the image recognition unit 24, the measurement line generation unit 25, the measurement reference point setting unit 27, the display form setting unit 28, and the apparatus controller 29.

The transducer array 11 of the ultrasound probe 1 includes a plurality of ultrasound transducers that are one-dimensionally or two-dimensionally arranged. In accordance with a drive signal supplied from the transmission/reception circuit 12, each of the ultrasound transducers transmits ultrasound and receives an ultrasound echo from a subject to output a signal based on the ultrasound echo. Each ultrasound transducer is configured by, for example, forming electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 31, which is composed of the transmission/reception circuit 12 and the image generation unit 21, acquires ultrasound images of a plurality of frames as a moving image in which a heart of the subject is imaged, by transmitting and receiving ultrasound beams using the ultrasound probe 1.

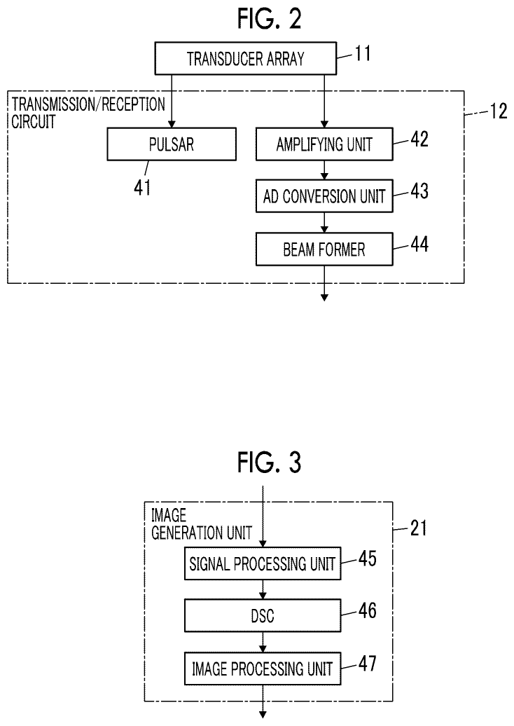

The transmission/reception circuit 12 transmits the ultrasound waves from the transducer array 11 and generates a sound ray signal based on reception signals acquired by the transducer array 11 under control of the apparatus controller 29. As shown in FIG. 2, the transmission/reception circuit 12 includes a pulsar 41 connected to the transducer array 11, and an amplification unit 42, an analog-to-digital (AD) conversion unit 43, and a beam former 44 that are sequentially connected in series to the transducer array 11.

The pulsar 41 includes, for example, a plurality of pulse generators, adjusts a delay amount of each drive signal based on a transmission delay pattern selected in accordance with a control signal from the apparatus controller 29 so that the ultrasound waves transmitted from the plurality of ultrasound oscillators of the transducer array 11 form an ultrasound beam, and supplies each drive signal to the plurality of ultrasound oscillators. As described above, in a case in which a pulsed or continuous wave-like voltage is applied to the electrodes of the ultrasound transducer of the transducer array 11, the piezoelectric material expands and contracts to generate pulsed or continuous wave-like ultrasound from each of the ultrasound transducers, whereby the ultrasound beam is formed from the combined wave of the ultrasound.

The transmitted ultrasound beam is, for example, reflected by a target such as a part of the subject and propagates toward the transducer array 11 of the ultrasound probe 1. The ultrasound echo propagating toward the transducer array 11 in this way is received by each of the ultrasound transducers constituting the transducer array 11. In such a case, each of the ultrasound transducers constituting the transducer array 11 receives the propagating ultrasound echo to expand and contract, generates the reception signal, which is an electrical signal, and outputs these reception signals to the amplification unit 42.

The amplification unit 42 amplifies the signal input from each of the ultrasound transducers constituting the transducer array 11 and transmits the amplified signal to the AD conversion unit 43. The AD conversion unit 43 converts the signal transmitted from the amplification unit 42 into digital reception data. The beam former 44 performs so-called reception focus processing by applying and adding the delay to each reception data received from the AD conversion unit 43. By the reception focus processing, each reception data, which is converted by the AD conversion unit 43, is phase-added, and the sound ray signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series.

The signal processing unit 45 corrects attenuation by distance of the sound ray signal received from the transmission/reception circuit 12 in accordance with depths of reflection positions of the ultrasound waves using a sound speed value set by the apparatus controller 29 and then performs envelope detection processing on the sound ray signal to generate a B-mode image signal that is tomographic image information related to tissues inside the subject.

The DSC 46 converts (raster-converts) the B-mode image signal, which is generated by the signal processing unit 45, into the image signal in accordance with a normal television signal scanning method.

The image processing unit 47 performs various types of necessary image processing such as gradation processing on the B-mode image signal input from the DSC 46, and then transmits the B-mode image signal to the display controller 22 and the image recognition unit 24. Hereinafter, the B-mode image signal, which is image-processed by the image processing unit 47, will be referred to as an ultrasound image.

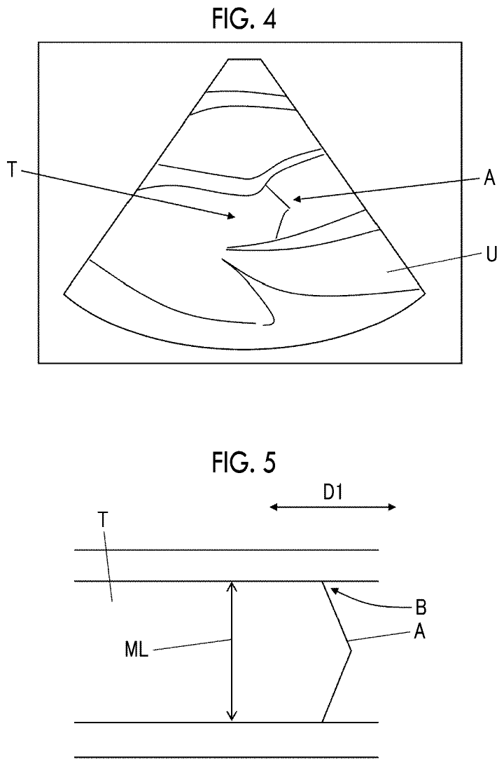

In the present invention, the image acquisition unit 31 acquires the ultrasound image in which the heart of the subject including a so-called left ventricular outflow tract is imaged. For example, as shown in FIG. 4, an ultrasound image U representing a so-called parasternal left ventricular long-axis cross section that vertically cuts a so-called aortic valve annulus of the heart is acquired. In the ultrasound image U representing the parasternal left ventricular long-axis cross section, a left ventricular outflow tract T and an aortic valve annulus A are usually included.

A technique of calculating a so-called cardiac output by capturing the ultrasound image U representing the tomographic plane of the heart of the subject using the ultrasound diagnostic apparatus and analyzing the captured ultrasound image U is known. The cardiac output is usually calculated by a calculation step of (1) measuring a diameter of a left ventricular outflow tract in the ultrasound image U of a frame representing a so-called parasternal left ventricular long-axis cross section at a mid-systolic phase of the heart to calculate a cross-sectional area of the left ventricular outflow tract T, (2) calculating a velocity time integral value of blood flow in the left ventricular outflow tract T with respect to a so-called apical five-chamber cross section or a so-called apical three-chamber cross section by a so-called pulse Doppler method, (3) calculating a so-called stroke volume by multiplying the cross-sectional area of the left ventricular outflow tract T by the velocity time integral value of the blood flow in the left ventricular outflow tract T, and (4) calculating the cardiac output by multiplying the stroke volume by a heart rate.

In a case in which the diameter of the left ventricular outflow tract T is measured in the calculation of the cardiac output, a measurement line may be disposed on the left ventricular outflow tract T in the ultrasound image U, and a length of the measurement line may be measured as the diameter of the left ventricular outflow tract T. The appropriate range for the disposition position of the measurement line is often determined by a guideline of an examination predetermined by a public interest organization such as an academic society or a guideline predetermined by institutional guidelines of a facility such as a hospital in which the examination is performed, and is often set based on a position of a characteristic anatomical structure of the aortic valve annulus A and like. Further, by the established guideline, a specific position within the appropriate range may be defined as the optimal position of the measurement line.

The image recognition unit 24 performs image recognition of a local anatomical structure of the heart in each of the plurality of frames acquired by the image acquisition unit 31. Here, the local anatomical structure includes any of the aortic valve annulus A, a mitral valve, or a left ventricle. The image recognition unit 24 can perform image recognition of the local anatomical structure by, for example, a so-called template matching method of searching for the ultrasound image U using template image data representing a general image or the like representing the local anatomical structure of the heart, which is stored in advance. The image recognition unit 24 can also perform image recognition of the local anatomical structure by, for example, inputting the ultrasound image U to a trained model in so-called machine learning in which the local anatomical structure of the heart is trained in advance.

In addition, the image recognition unit 24 performs image recognition of the left ventricular outflow tract T by performing image analysis on the ultrasound image U, and specifies a traveling direction of the left ventricular outflow tract T subjected to the image recognition by using a so-called thinning algorithm or the like.

As schematically shown in FIG. 5, the measurement line generation unit 25 generates a measurement line ML disposed on the ultrasound image U for measuring the diameter of the left ventricular outflow tract T, that is, the left ventricular outflow tract diameter. The measurement line generation unit 25 can generate the measurement line ML to extend in a direction perpendicular to the traveling direction D1 of the left ventricular outflow tract T specified by the image recognition unit 24, for example. In addition, the measurement line generation unit 25 can generate the measurement line ML at a position on the left ventricular outflow tract T in the ultrasound image U designated by the user via the input device 30, for example. In this case, the user designates the disposition position of the measurement line ML with reference to the appropriate range related to the position of the measurement line ML determined by the established guideline based on, for example, a position of a valve cusp B of the aortic valve annulus A, a position of the mitral valve, or a position of a boundary between the left ventricle and the aorta.

The memory 26 stores the appropriate range related to the position of the measurement line ML in advance. The appropriate range includes a first appropriate range defined by the established guideline and a second appropriate range reflecting the preference of the user. The first appropriate range is set as a range from the position of the valve cusp B of the aortic valve annulus A to a position that is separated from the position by a predetermined distance, such as 1.0 cm, on the left ventricle side along the traveling direction D1, for example. The first appropriate range and the second appropriate range can be input in advance by the user via the input device 30, for example. In addition, the second appropriate range can also be determined by, for example, the apparatus controller 29 based on the position of the measurement line ML disposed by the user in the past examination.

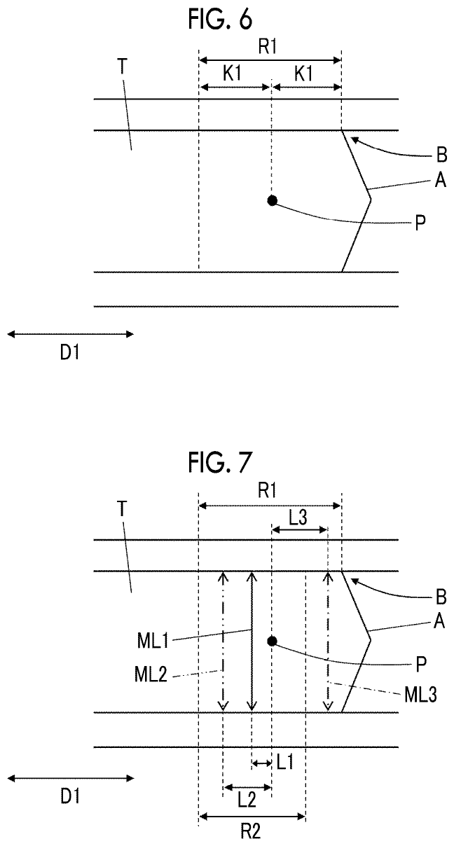

The measurement reference point setting unit 27 sets a measurement reference point P in the appropriate range in the ultrasound image U, for example, as shown in FIG. 6, based on the predetermined appropriate range of the measurement line ML stored in the memory 26 and the local anatomical structure subjected to the image recognition by the image recognition unit 24. The measurement reference point setting unit 27 can set the measurement reference point P based on, for example, the first appropriate range R1 and the local anatomical structure subjected to the image recognition. In the established guideline, for example, in a case in which a midpoint of the first appropriate range R1 is defined as the optimal position of the measurement line ML, the measurement reference point setting unit 27 can set, as the measurement reference point P, a point that is separated from the midpoint of the first appropriate range R1, that is, the position of the valve cusp B by a distance K1 that is a length of half of the first appropriate range R1 on the left ventricle side along the traveling direction D1. The measurement reference point setting unit 27 can store in advance, for example, an anatomical positional relationship between the position of the mitral valve and the position of the boundary between the left ventricle and the aorta and the first appropriate range R1, and can also set the measurement reference point P in the first appropriate range R1 based on the position of the mitral valve or the position of the boundary between the left ventricle and the aorta, based on the established guideline.

In addition, the measurement reference point setting unit 27 can also reflect the preference of the user based on past measurement results, and set the measurement reference point P based on the second appropriate range and the local anatomical structure subjected to the image recognition, for example. In this case, the measurement reference point setting unit 27 can set, for example, a midpoint of the second appropriate range in the traveling direction D1 of the left ventricular outflow tract T as the measurement reference point P.

The display form setting unit 28 sets the display form of the measurement line ML generated by the measurement line generation unit 25 based on the positional relationship between the measurement line ML and the measurement reference point P set by the measurement reference point setting unit 27. The display form setting unit 28 can set, as the display form of the measurement line ML, for example, a display form in which at least one of a color of the measurement line ML, a type of a line constituting the measurement line ML such as a solid line and a dotted line, a shape of the measurement line ML, or a transmittance of the measurement line ML is changed. For example, as shown in FIG. 7, the display form setting unit 28 can set the display forms of a measurement line ML1, a measurement line ML2, and a measurement line ML3 to be different from each other. The measurement line ML1 is separated from the measurement reference point P by a distance L1, the measurement line ML2 is separated from the measurement reference point P by a distance L2 longer than the distance L1, and the measurement line ML3 is separated from the measurement reference point P by a distance L3 longer than the distance L2, in the traveling direction D1 of the left ventricular outflow tract T.

In addition, in a case in which the measurement reference point P is set based on the first appropriate range R1 and the local anatomical structure such as the aortic valve annulus A, the display form setting unit 28 can also set the display form of the measurement line ML based on whether or not the measurement line ML is within the second appropriate range R2, in addition to the distance from the measurement reference point P to the measurement line ML. For example, in the example of FIG. 7, the measurement line ML1 and the measurement line ML2 are within the second appropriate range R2, and the measurement line ML3 is outside the second appropriate range R2. In this case, the display form setting unit 28 can set colors of the measurement lines ML1, ML2, and ML3 depending on the distance from the measurement reference point P in the traveling direction D1, and can set the types of the lines to be different between the measurement lines ML1 and ML2 within the second appropriate range R2 and the measurement line ML3 outside the second appropriate range R2. In this way, the display form setting unit 28 can set the display form of the measurement line ML set according to the distance from the measurement reference point P in the traveling direction D1 and the display form of the measurement line ML set according to whether or not the measurement line ML is within the second appropriate range R2, respectively.

The display controller 22 performs predetermined processing on the ultrasound image U or the like acquired by the image acquisition unit 31, and displays the processed ultrasound image U or the like on the monitor 23, under the control of the apparatus controller 29. In addition, the display controller 22 displays the measurement line ML on the monitor 23 in accordance with the display form set by the display form setting unit 28.

The monitor 23 displays the ultrasound image U or the like under the control of the display controller 22 and includes, for example, a display device such as a liquid crystal display (LCD) or an organic electroluminescence display (organic EL display).

The input device 30 is an input device for the user to perform an input operation, and is configured by, for example, a device such as a keyboard, a mouse, a trackball, a touchpad, and a touch sensor disposed in a state of being superimposed on the monitor 23.

In the present embodiment, each processing performed by the processor 32 is executed by any computer. Moreover, any computer may execute these processes by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system capable of executing each processing.

The processor 32 may be composed of one or a plurality of pieces of hardware, and types of hardware are not limited. For example, the processor 32 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphics processing unit (GPU), or a neural processing unit (NPU). Types of hardware may be a combination of different types of hardware. In a case where a plurality of pieces of hardware are configured to execute one or a plurality of types of processing of a processor, the plurality of pieces of hardware may be present in apparatuses physically separated from each other or may be present in the same apparatus. Further, in any of the embodiments, the order of each processing performed by the processor 32 is not limited to the above-described order, and may be changed as appropriate. The hardware is composed of an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

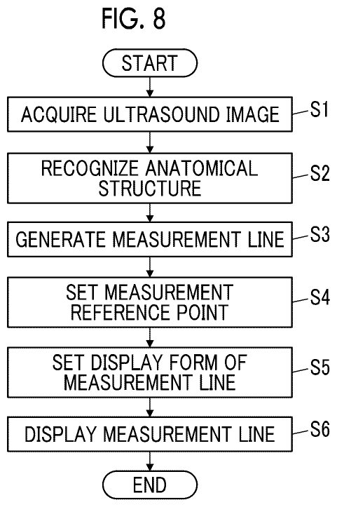

Next, an operation of the ultrasound diagnostic apparatus according to First Embodiment will be described with reference to the flowchart shown in FIG. 8.

In step S1, the image acquisition unit 31 generates the ultrasound image U in which the heart of the subject representing, for example, the parasternal left ventricular long-axis cross section including the left ventricular outflow tract T is imaged. In such a case, under the control of the apparatus controller 29, the transmission and reception of the ultrasound from the plurality of transducers of the transducer array 11 are started in accordance with the drive signal from the pulsar 41 of the transmission/reception circuit 12 of the ultrasound probe 1, the ultrasound echo from the subject is received by the plurality of transducers of the transducer array 11, and the reception signal as the analog signal is output to the amplification unit 42, is amplified, and then is subjected to the AD conversion via the AD conversion unit 43 to acquire the reception data.

The reception focus processing is performed on the reception data by the beam former 44, the sound ray signal generated by the reception focusing processing is transmitted to the image generation unit 21 of the apparatus main body 2, and thus the ultrasound image U representing the heart of the subject is generated by the image generation unit 21. In this case, the signal processing unit 45 of the image generation unit 21 performs the correction of the attenuation in accordance with the depth of the reflection position of the ultrasound and the envelope detection processing on the sound ray signal, the DSC 46 performs the conversion into the image signal in accordance with the normal television signal scanning method, and the image processing unit 47 performs various types of necessary image processing, such as gradation processing. The ultrasound image U generated in step S1 as described above is transmitted to the display controller 22 and the image recognition unit 24.

In step S2, the image recognition unit 24 recognizes the local anatomical structure of the heart such as the aortic valve annulus A, the mitral valve, and the left ventricle in the ultrasound image U acquired in step S1. The image recognition unit 24 can recognize the local anatomical structure of the heart by, for example, a method of template matching, a method using a trained model in machine learning, or the like.

In addition, the image recognition unit 24 performs image recognition of the left ventricular outflow tract T in the ultrasound image U acquired in step S1, and specifies the traveling direction D1 of the left ventricular outflow tract T by a thinning algorithm or the like.

In step S3, the measurement line generation unit 25 generates, for example, as shown in FIG. 5, the measurement line ML disposed in the left ventricular outflow tract T shown in the ultrasound image U acquired in step S1. The measurement line generation unit 25 can generate the measurement line ML to extend in a direction perpendicular to the traveling direction D1 of the left ventricular outflow tract T specified in step S2 and at a position on the ultrasound image U designated by the user via the input device 30, for example.

In step S4, the measurement reference point setting unit 27 sets the measurement reference point P within the appropriate range in the ultrasound image U, for example, as shown in FIG. 6, based on the predetermined appropriate range of the measurement line ML stored in the memory 26 and the local anatomical structure subjected to the image recognition in step S2. In the established guideline, for example, in a case in which a midpoint of the first appropriate range R1 is defined as the optimal position of the measurement line ML, the measurement reference point setting unit 27 can set the midpoint of the first appropriate range R1 as the measurement reference point P.

In step S5, the display form setting unit 28 sets the display form of the measurement line ML generated in step S3 based on the positional relationship of the measurement line ML with respect to the measurement reference point P set in step S4. The display form of the measurement line ML includes a color of the measurement line ML, a type of a line constituting the measurement line ML such as a solid line and a dotted line, a shape of the measurement line ML, a transmittance of the measurement line ML, and the like. For example, as shown in FIG. 7, the display form setting unit 28 can set the display forms of a measurement line ML1, a measurement line ML2, and a measurement line ML3 to be different from each other. The measurement line ML1 is separated from the measurement reference point P by a distance L1, the measurement line ML2 is separated from the measurement reference point P by a distance L2 longer than the distance L1, and the measurement line ML3 is separated from the measurement reference point P by a distance L3 longer than the distance L2, in the traveling direction D1 of the left ventricular outflow tract T.

In addition, in a case in which the measurement reference point P is set based on the first appropriate range R1 and the local anatomical structure such as the aortic valve annulus A, the display form setting unit 28 can also set the display form of the measurement line ML based on whether or not the measurement line ML is within the second appropriate range R2, in addition to the distance from the measurement reference point P to the measurement line ML. For example, the display form setting unit 28 can set the display form of the measurement line ML set according to the distance from the measurement reference point P in the traveling direction D1 and the display form of the measurement line ML set according to whether or not the measurement line ML is within the second appropriate range R2, respectively.

In step S6, the display controller 22 displays the measurement line ML on the monitor 23 in accordance with the display form set in step S5. The user can easily understand the appropriateness of the disposition position of the measurement line ML in the left ventricular outflow tract T by checking the display form of the measurement line ML on the monitor 23, and can easily dispose the measurement line ML at the optimal position within the appropriate range. As a result, for example, the variation in the disposition position of the measurement line ML for each user or each examination can be reduced, and the diameter of the left ventricular outflow tract T can be accurately measured according to the same reference.

In a case in which the processing of step S6 is completed in this manner, the operation of the ultrasound diagnostic apparatus according to the flowchart of FIG. 8 is completed.

As described above, according to the ultrasound diagnostic apparatus of First Embodiment of the present invention, the image recognition unit 24 performs image recognition of the local anatomical structure of the heart from the ultrasound image U, the measurement reference point setting unit 27 sets the measurement reference point P within the appropriate range in the ultrasound image U based on the predetermined appropriate range of the measurement line ML and the local anatomical structure subjected to the image recognition by the image recognition unit 24, the display form setting unit 28 sets the display form of the measurement line ML generated by the measurement line generation unit 25 based on the positional relationship with respect to the measurement reference point P set by the measurement reference point setting unit 27, and the display controller 22 displays the measurement line ML on the monitor 23 in accordance with the display form of the measurement line ML set by the display form setting unit 28. Thereby, the user can accurately and easily dispose the measurement line ML at the appropriate position.

A case has been described in which the transmission/reception circuit 12 is provided in the ultrasound probe 1, but the transmission/reception circuit 12 may be provided in the apparatus main body 2.

Further, a case has been described in which the image generation unit 21 is provided in the apparatus main body 2, but the image generation unit 21 may be provided in the ultrasound probe 1.

The apparatus main body 2 may be a so-called stationary type, a portable type that is easily carried, or a so-called handheld type that is configured by, for example, a smartphone or a tablet type computer. In this way, the type of the device constituting the apparatus main body 2 is not particularly limited.

It should be noted that, although the measurement line generation unit 25 generates the measurement line ML at the position designated by the user in the ultrasound image U, the method of generating the measurement line ML is not particularly limited thereto. The measurement line generation unit 25 can also generate the measurement line ML based on the local anatomical structure of the heart subjected to the image recognition by the image recognition unit 24, for example. In this case, the measurement line generation unit 25 can dispose the measurement line ML at a position that is advanced by a predetermined distance on the left ventricle side in the traveling direction D1 of the left ventricular outflow tract T from the position of the valve cusp B of the aortic valve annulus A, for example. The predetermined distance from the position of the valve cusp B to the measurement line ML can be set by the user in advance via the input device 30, and can also be automatically set, for example, by an average value in a plurality of examinations based on the distance from the position of the valve cusp B to the measurement line ML set by the user in the past examination.

The measurement line generation unit 25 can also generate a plurality of measurement lines ML. In this case, the measurement line generation unit 25 can generate a plurality of measurement lines ML arranged at equal intervals in a range from the position of the valve cusp B of the aortic valve annulus A to a position that is advanced by a predetermined distance on the left ventricle side in the traveling direction D1 of the left ventricular outflow tract T, for example. In addition, the measurement line generation unit 25 can also generate a plurality of measurement lines ML arranged at equal intervals within a certain range including the position on the ultrasound image U designated by the user via the input device 30, for example. The interval between the plurality of measurement lines ML and the length of the certain range in which the plurality of measurement lines ML are arranged in the traveling direction D1 can be input in advance by the user via the input device 30, for example.

In a case in which a plurality of measurement lines ML are generated in this way, the display form setting unit 28 can set the display forms of the plurality of measurement lines ML based on the positional relationship between the measurement reference point P set by the measurement reference point setting unit 27 and each of the plurality of measurement lines ML. The display controller 22 can display the plurality of measurement lines ML on the monitor 23 in accordance with the set display form. The user can select, via the input device 30, one measurement line ML that is optimal for the first appropriate range R1 and the second appropriate range R2 while checking the display form of the plurality of measurement lines ML displayed on the monitor 23, for example.

Although it is described that the measurement reference point setting unit 27 sets the measurement reference point P after the measurement line generation unit 25 generates the measurement line ML. However, the measurement line generation unit 25 can also generate the measurement line ML after the measurement reference point setting unit 27 sets the measurement reference point P. Even in this case, the display form setting unit 28 can set the display form of the measurement line ML based on the positional relationship between the measurement reference point P and the measurement line ML.

The display form setting unit 28 can also display whether or not the generated measurement line ML is within the first appropriate range R1 and whether or not the generated measurement line ML is within the second appropriate range R2 on the monitor 23 in a form of a message by, for example, so-called pop-up display or the like.

In addition, the display form setting unit 28 can calculate the appropriateness of the disposition position of the measurement line ML based on the positional relationship between the measurement reference point P and the measurement line ML, and can display the calculated appropriateness on the monitor 23 in association with the measurement line ML. In this case, the display form setting unit 28 can calculate the appropriateness such that the value is higher as the disposition position of the measurement line ML is closer to the measurement reference point P and the value is lower as the disposition position of the measurement line ML is farther from the measurement reference point P, for example, by calculating an inverse of a distance between the measurement reference point P and the measurement line ML as the appropriateness. The display form setting unit 28 can also weight the appropriateness by, for example, multiplying the appropriateness by a predetermined ratio that is greater than 0 and less than 1 in a case in which the measurement line ML is disposed outside the second appropriate range R2 reflecting the preference of the user.

In addition, the user can appropriately change the first appropriate range R1 defined by the established guideline and the second appropriate range R2 reflecting the preference of the user via the input device 30. As a result, in a case in which a regulation in the established guideline is changed, the first appropriate range and the second appropriate range are changed to correspond to a case in which the preference of the user is changed.

### SECOND EMBODIMENT

In general, in a facility or the like in which a plurality of technicians or doctors are employed, one ultrasound diagnostic apparatus is used by a plurality of users. Therefore, the ultrasound diagnostic apparatus can also set the appropriate range related to the disposition position of the measurement line ML for each user.

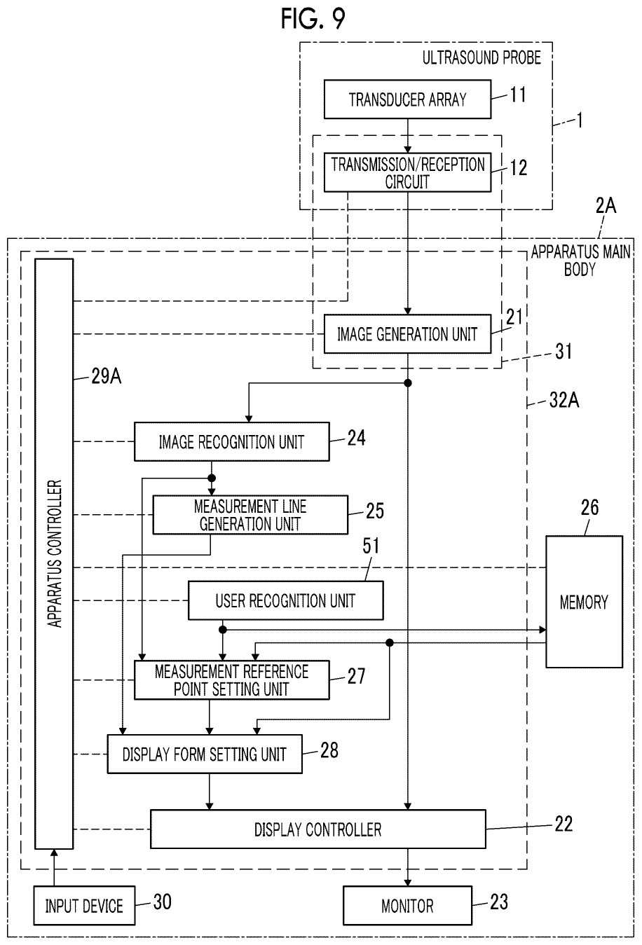

FIG. 9 shows a configuration of an ultrasound diagnostic apparatus according to Second Embodiment. The ultrasound diagnostic apparatus according to Second Embodiment comprises an apparatus main body 2A instead of the apparatus main body 2, as compared with the ultrasound diagnostic apparatus according to First Embodiment shown in FIG. 1. The apparatus main body 2A is obtained by further providing a user recognition unit 51 to the apparatus main body 2 in the first embodiment, and including an apparatus controller 29A instead of the apparatus controller 29.

In the apparatus main body 2A, the user recognition unit 51 is connected to the memory 26 and the measurement reference point setting unit 27. The user recognition unit 51 is connected to the apparatus controller 29A. In addition, a processor 32A for the apparatus main body 2A is configured by the image generation unit 21, the display controller 22, the image recognition unit 24, the measurement line generation unit 25, the measurement reference point setting unit 27, the display form setting unit 28, and the apparatus controller 29A.

The user recognition unit 51 recognizes the user based on, for example, user identification information input by the user via the input device 30. The user identification information includes, for example, a unique identifier (ID) of the user. The information on the user recognized by the user recognition unit 51 is transmitted to the memory 26 and the measurement reference point setting unit 27.

The memory 26 stores a plurality of appropriate ranges corresponding to a plurality of users. That is, the memory 26 stores the first appropriate range R1 and the second appropriate range R2 related to the disposition position of the measurement line ML for each user recognized by the user recognition unit 51. The memory 26 can store the set first appropriate range R1 and second appropriate range R2 in association with the recognized user in a state in which a specific user is recognized by the user recognition unit 51, for example, by setting the first appropriate range R1 and the second appropriate range R2 by an input from the user via the input device 30.

The measurement reference point setting unit 27 reads out the appropriate range corresponding to the user recognized by the user recognition unit 51 from the memory 26, and sets the measurement reference point P based on the read appropriate range.

The display form setting unit 28 sets the display form of the measurement line ML based on the positional relationship between the appropriate range read out for each user by the measurement reference point setting unit 27 and the measurement line ML generated by the measurement line generation unit 25. The display controller 22 displays the measurement line ML on the monitor 23 in accordance with the display form set by the display form setting unit 28.

As described above, according to the ultrasound diagnostic apparatus of Second Embodiment of the present invention, the measurement reference point P is set by using the appropriate range stored for each user recognized by the user recognition unit 51, and the display form of the measurement line ML is set based on the set measurement reference point P. Therefore, even in a case in which the user who uses the ultrasound diagnostic apparatus is changed, the user who uses the ultrasound diagnostic apparatus can accurately and easily dispose the measurement line ML at an appropriate position with respect to the appropriate range corresponding to the user.

The input device 30 can include a biometric authentication sensor such as a so-called fingerprint authentication sensor that reads a feature of a fingerprint of the user, and a face authentication sensor that captures an optical image of a face of the user and reads a feature of the face of the user by analyzing the optical image. In this case, the user recognition unit 51 can recognize the user by using the biometric authentication by the input device 30.

### EXPLANATION OF REFERENCES

1: ultrasound probe
2, 2A: apparatus main body
11: transducer array
12: transmission/reception circuit
21: image generation unit
22: display controller
23: monitor
24: image recognition unit
25: measurement line generation unit
26: memory
27: measurement reference point setting unit
28: display form setting unit
29, 29A: apparatus controller
30: input device
31: image acquisition unit
32, 32A: processor
41: pulsar
42: amplification unit
43: AD conversion unit
44: beam former
45: signal processing unit
46: DSC
47: image processing unit
51: user recognition unit
A: aortic valve annulus
B: valve cusp
D1: traveling direction
K1, L1, L2, L3: distance
ML, ML1, ML2, ML3: measurement line
P: measurement reference point
R1: first appropriate range
R2: second appropriate range
T: left ventricular outflow tract
U, U1, U2: ultrasound image

What is claimed is:

1. An ultrasound diagnostic apparatus that displays a measurement line of a left ventricular outflow tract diameter in an ultrasound image in which a heart of a subject is imaged, the ultrasound diagnostic apparatus comprising:
a monitor; and
a processor configured to:
perform image recognition of a local anatomical structure of the heart from the ultrasound image;
generate the measurement line;
set a measurement reference point within a predetermined appropriate range of the measurement line in the ultrasound image based on the appropriate range and the local anatomical structure;
set a display form of the measurement line based on a positional relationship with respect to the measurement reference point;
display the measurement line on the monitor in accordance with the display form.

2. The ultrasound diagnostic apparatus according to claim 1, further comprising:
a memory configured to store the appropriate range.

3. The ultrasound diagnostic apparatus according to claim 1,
wherein the appropriate range is a range reflecting a preference of a user or a range defined by an established guideline.

4. The ultrasound diagnostic apparatus according to claim 2,
wherein the appropriate range is a range reflecting a preference of a user or a range defined by an established guideline.

5. The ultrasound diagnostic apparatus according to claim 1,
wherein the processor is configured to perform image recognition of any of an aortic valve, a mitral valve, and a left ventricle as the local anatomical structure.

6. The ultrasound diagnostic apparatus according to claim 2,
wherein the processor is configured to perform image recognition of any of an aortic valve, a mitral valve, and a left ventricle as the local anatomical structure.

7. The ultrasound diagnostic apparatus according to claim 3,
wherein the processor is configured to perform image recognition of any of an aortic valve, a mitral valve, and a left ventricle as the local anatomical structure.

8. The ultrasound diagnostic apparatus according to claim 1,
wherein the processor is configured to generate the measurement line based on the local anatomical structure.

9. The ultrasound diagnostic apparatus according to claim 2,
wherein the processor is configured to generate the measurement line based on the local anatomical structure.

10. The ultrasound diagnostic apparatus according to claim 3,
wherein the processor is configured to generate the measurement line based on the local anatomical structure.

11. The ultrasound diagnostic apparatus according to claim 4,
wherein the processor is configured to generate the measurement line based on the local anatomical structure.

12. The ultrasound diagnostic apparatus according to claim 1,
wherein the processor is configured to set a midpoint of the appropriate range as the measurement reference point.

13. The ultrasound diagnostic apparatus according to claim 2,
wherein the processor is configured to set a midpoint of the appropriate range as the measurement reference point.

14. The ultrasound diagnostic apparatus according to claim 1,
wherein the processor is configured to set the measurement reference point in accordance with a preference of a user based on a past measurement result.

15. The ultrasound diagnostic apparatus according to claim 1,
wherein the processor is configured to set a display form in which at least one of a color, a shape, or a type of the measurement line is changed depending on the positional relationship of the measurement line with respect to the measurement reference point.

16. The ultrasound diagnostic apparatus according to claim 1,
wherein the processor is configured to set the measurement reference point based on the appropriate range input by a user and the local anatomical structure.

17. The ultrasound diagnostic apparatus according to claim 2,
wherein the memory stores a plurality of appropriate ranges corresponding to a plurality of users, and
the processor is configured to:
recognize a user in a current examination;
read out the appropriate range corresponding to the recognized user from the memory; and
set the measurement reference point based on the read appropriate range.

18. The ultrasound diagnostic apparatus according to claim 17,
wherein the processor is configured to recognize the user based on user identification information input by the user.

19. The ultrasound diagnostic apparatus according to claim 17,
wherein the processor is configured to recognize the user by using biometric authentication.

20. A control method of an ultrasound diagnostic apparatus that displays a measurement line of a left ventricular outflow tract diameter in an ultrasound image in which a heart of a subject is imaged, the control method comprising:
performing image recognition of a local anatomical structure of the heart from the ultrasound image;
generating the measurement line;
setting a measurement reference point within a predetermined appropriate range of the measurement line in the ultrasound image based on the appropriate range and the local anatomical structure subjected to the image recognition;
setting a display form of the measurement line based on a positional relationship with respect to the measurement reference point; and
displaying the measurement line on a monitor in accordance with the set display form.
